(19) United States
(12) Patent Application Publication
(10) Pub. No.: US 2010/0173421 A1
(43) Pub. Date: Jul. 8, 2010

(54) SENSOR

(75) Inventors: Sergey Anatoliyovich Piletsky, Bedforshire (GB); Yves Frederic Henry, Concarneau (FR); Khalku Karim, Cambridgeshire (GB); Peter Georg Laitenberger, Cambridgeshire (GB)

Correspondence Address: STITES & HARBISON PLLC, 1199 NORTH FAIRFAX STREET, SUITE 900, ALEXANDRIA, VA, 22314, US

(73) Assignees: CRANFIELD UNIVERSITY, Bedfordshire (GB); SPHERE MEDICAL LIMITED, Cambridgeshire (GB)

(21) Appl. No.: 12/529,579

(22) PCT Filed: Feb. 29, 2008
(86) PCT No.: PCT/GB2008/000697

§ 371 Date: Feb. 2, 2010

(30) Foreign Application Priority Data
Mar. 3, 2007 (GB) 0704151.0

(51) Int. Cl. G01N 33/50

(52) U.S. Cl. 436/131

(57) ABSTRACT

This invention relates to a sensor and in particular to a sensor for the detection of biologically important species. Specifically, the invention provides a method for detecting an analyte in the presence of at least one interferent in a sample. The method comprises the steps of providing a sensor having a transducer and a receptor layer in communication with the transducer, in which the receptor layer comprises a material for absorbing the analyte; exposing the receptor layer to the sample; treating the receptor layer to remove selectively the at least one interferent; and measuring the signal from the transducer. The treatment step is performed by applying a change in potential, a change in pH or a change in temperature to the receptor layer, by washing the receptor layer, by irradiating the receptor layer, or a combination thereof.

### TECHNICAL FIELD

This invention relates to a sensor and in particular to a sensor for the detection of biologically important species.

### BACKGROUND ART

Modern healthcare relies extensively on a range of chemical and biochemical analytical tests on a variety of bodily fluids to enable diagnosis and management of disease. Medical and technological advances have considerably expanded the scope of diagnostic testing over the past few decades. Moreover, an increasing understanding of the human body, together with the emergence of developing technologies, such as microsystems technology and nanotechnology, are expected to have a profound impact on diagnostic technology.

Increasingly, diagnostic tests in hospitals are carried out at the point-of-care (PoC), in particular, in situations, where a rapid response is a prime consideration and therapeutic decisions have to be made quickly. Despite recent advances in PoC testing, several compelling needs remain unmet. Many of the presently available diagnostic tests rely on the use of sophisticated biological receptors, such as enzymes, antibodies and DNA. Due to their biological derivation, these biomolecules typically suffer from a number of limitations when used in sensing applications, for example, poor reproducibility, instability during manufacture, sensitivity to environmental factors, such as pH, ionic strength, temperature etc., and problems associated with the sterilisation process.

A promising route to overcome these issues is offered by synthetic polymer-based receptors, such as molecularly imprinted polymers (MIPs). Synthetic receptors avoid many of the disadvantages associated with biological receptors. Molecular imprinting, for example, is a generic and cost-effective technique for preparing synthetic receptors, which combine high affinity and high specificity with robustness and low manufacturing costs. In addition, MIP receptor materials have already been demonstrated for a wide range of clinically relevant compounds and diagnostic markers. In contrast to biological receptors, synthetic receptors, and particularly MIPs, typically are stable at low and high pH, pressure and temperature, are inexpensive and easy to prepare, tolerate organic solvents, may be prepared for practically any analyte, and are compatible with micromachining and microfabrication technology.

Molecular imprinting may be defined as the process of template-induced formation of specific recognition sites (binding or catalytic) in a material, where the template directs the positioning and orientation of the material's structural components by a self-assembling mechanism. The material itself could be oligomeric, polymeric (for example, organic MIPs and inorganic imprinted silica gels) or two-dimensional surface assemblies (grafted monolayers).

In many applications, for example, where the receptor is to be used repeatedly without significant regeneration between applications, the use of so-called non-covalent MIPs is generally preferred, in particular in sensing applications. As the template/analyte is only weakly bound by non-covalent interactions to these receptor materials, it can be relatively easily removed from the synthetic receptor and the sensor regenerated for a new measurement. In general, non-covalent imprinting is easier to achieve and applicable to a wider spectrum of templates.

In non-covalent MIPs, the monomer(s) contained within the polymer interact(s) with the template through non-covalent interactions, for example, hydrogen bonding, electrostatic interaction, coordination-bond formation etc. FIG. 1 shows a schematic representation of the self-assembly of a MIP from monomeric starting materials to form a polymer having binding sites with specificity for the template, i.e. the target analyte or a structural analogue thereof, and the subsequent elution or extraction of the template.

This technique has been employed to create successfully MIPs for a range of chemical compounds, ranging from small molecules (up to 1200 Da), such as small organic molecules (e.g. glucose) and drugs, to large proteins and cells. The resulting polymers are robust, inexpensive and, in many cases, possess affinity and specificity that is suitable for diagnostic applications. The high specificity and stability of MIPs render them promising alternatives to enzymes, antibodies, and natural receptors for use in sensor technology. See WO 2005/075995 for further details regarding MIPs and other synthetic polymers.

Although specific to the target, a selective chemical receptor will also interact to some extent with other compounds present in complex sample mixture, such as blood. For example, uric acid and ascorbic acid are very often cited as strong interferents, when attempting to detect electrochemically a given compound; one particular example being the measurement of glucose in a blood sample of a human. In order to reduce the influence of the interferents on the measurements, it has been suggested (M. F. Jakeway et al, Anal. Chem. 1994, Nov. 15, 66 (22), 3882-8) to coat the recognition elements with a protective material that is able to repel the interferents and therefore prevent the interferents from reaching the electrochemical transducer. This approach may therefore help to reduce the electrochemical signal due to the interferents and enable the desired signal arising from the presence of the analyte which is to be detected to be measured, but can considerably reduce the sensitivity of the sensor and increase the response time of the sensor.

There remains a requirement in the art therefore for increased selectivity without unduly reducing sensitivity.

### DISCLOSURE OF INVENTION

Accordingly, the present invention provides a method for detecting an analyte in the presence of at least one interferent in a sample comprising the steps of

providing a sensor having a transducer and a receptor layer in communication with the transducer, wherein the receptor layer comprises a material for absorbing the analyte;

exposing the receptor layer to the sample;

treating the receptor layer to remove selectively the at least one interferent; and

measuring the signal from the transducer.

This provides a detection methodology which allows for the specific measurement of one compound present in a complex mixture without the use of protective coatings and loss of sensitivity using high binding affinity synthetic receptors immobilised on a microsensor.

When the sensor is an electrochemical sensor and the receptor layer is not intrinsically a good electrical conductor, an electrically conductive material may be dispersed throughout the receptor layer. This can facilitate electrical conduction between analyte in a binding site in the receptor layer and the bulk of the receptor layer, and hence between the analyte and the transducer itself. Suitable conductive materials are conductive particulate solids e.g. of metal (e.g. gold, silver, copper or platinum), of carbon (e.g. carbon black, fullerenes, nanotubes or spheres), and/or of conductive organic materials. Particulate solids may comprise powders, nanoparticles and wires. When the receptor layer comprises a polymer produced from a pre-polymer composition, the conductive material may be dispersed in this composition before it is polymerised to form the polymer.

### BRIEF DESCRIPTION OF DRAWINGS

The present invention will now be described with reference to the accompanying drawings in which:

FIG. 1 shows a schematic representation of the self-assembly of a MIP and is discussed hereinabove with reference to the state of the art;

FIG. 2 shows a sensor for performing the method of the present invention incorporating a MIP (in FIG. 2A), to which is presented a complex sample comprising of a number of interferents in addition to the analyte (FIG. 2B), followed by a washing step (FIG. 2C);

FIG. 3 shows the results of using the method of the present invention in three graphs (A-C); and

FIG. 4 shows a sensor for performing the method of the present invention incorporated into an intravenous monitoring system.

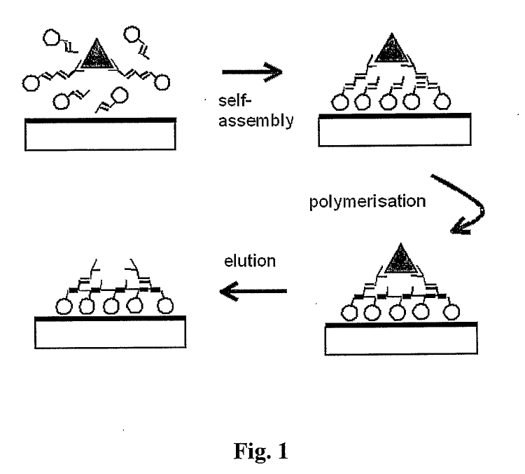

### MODES FOR CARRYING OUT THE INVENTION

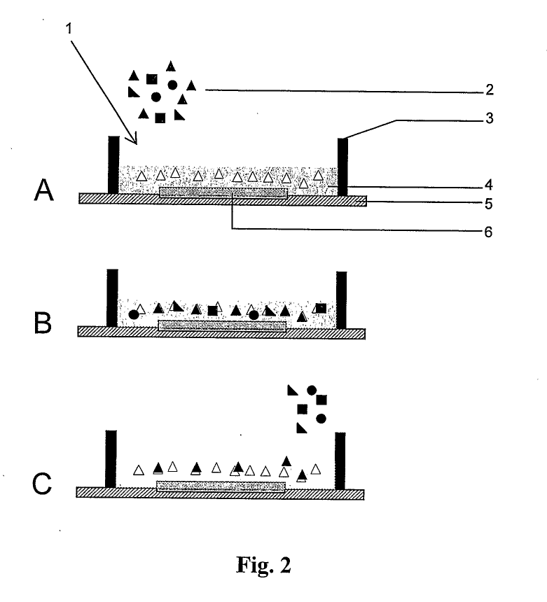

As shown in FIG. 2A, the sensor 1 is presented with a sample 2. The sample 2 is typically a fluid sample, preferably a liquid and most preferably a bodily fluid, such as blood. The sample is a “complex sample” in that it comprises the analyte being detected (represented in FIG. 2 by the equilateral triangles) as well as one or more interferents (represented by the squares, circles and right-angled triangles), which can interfere with the specific detection of the analyte. For example, the interferents may produce higher signal levels in the sensor in comparison to the analyte and/or be present in significantly higher levels and/or quench the activity of the analyte. It is therefore difficult, if not impossible, to determine the presence and measure the quantity of the target analyte in the sample.

An example is the electrochemical detection of the anaesthetic drug propofol in a complex sample of a patient's blood and containing other electroactive compounds, such as ascorbic acid and uric acid, as interferents.

In FIG. 2A, the sensor 1 comprises a confinement structure 3, a receptor layer 4, a substrate 5 and a transducer 6. Such a sensor is described in more detail in WO 2005/075995. The confinement structure 3 is disposed on the substrate 5. The confinement structure 3 comprises a first limiting structure defining a first interior space. As shown in FIG. 2A, the transducer 6 and the receptor layer 4 are disposed in the first interior space. The receptor layer 4 is on or proximal to the transducer 6 such that it is in communication with the transducer. Preferably the first limiting structure is a continuous structure, i.e. the walls are continuous and fully surround enclose the first interior space and most preferably is annular, i.e. a “well”. A second limiting structure defining a second interior space which encloses the first limiting structure may also be provided as described in WO 2005/075995. The first and second limiting structures are preferably composed of polyimide. The sensor 1 may further comprise a channel to contain the sample and to define a flow path to direct the sample to the receptor layer 4.

Any material having a high binding affinity and selectivity for the analyte and which may be immobilised on a microsensor chip may be used as the receptor layer 4. For example, the receptor layer 4 may comprise a synthetic polymer, a biomolecule or a combination thereof, more preferably the receptor layer comprises an ionophore, a molecularly imprinted polymer, an enzyme, an antigen, an imprinted silica gel, a two-dimensional surface assembly (grafted monolayers) or a combination thereof, more preferably the receptor layer 4 comprises a synthetic polymer and most preferably a MIP.

Suitable MIPs are described hereinabove and any of these MIPs may be incorporated in the receptor layer 4. By way of an example, where the analyte to be detected is propofol, the MIP is preferably a polymer based on one or more of the monomers N,N-diethylamino ethyl methacrylate (DEAEM), acrylamide, 2-(trifluoromethyl)acrylic acid (TFMAA), itaconic acid and ethylene glycol methacrylate phosphate (EGMP). The cross-linker is preferably selected from ethylene glycol dimethacrylate (EDMA), glycerol dimethacrylate (GDMA), trimethylacrylate (TRIM), divinylbenzene (DVB), methylenebisacrylamide and piperazinebisacrylamide (which are particularly suitable for cross linking acylamides), phenylene diamine, dibromobutane, epichlorohydrine, trimethylolpropane trimethacrylate and N,N′-methylenebisacrylamide. The mole ratio of monomer to cross-linker is preferably from 1:1 to 1:15. See WO 02/00737 and WO 2006/120381 for further details of propofol receptors.

The receptor layer 4 is in communication with a transducer 6. The transducer 6 may be an amperometric transducer, a potentiometric transducer, a conductimetric transducer, an optical (including fluorescent) transducer, a gravimetric transducer, a surface-acoustic wave transducer, a resonant transducer, a capacitive transducer or a thermal transducer. The receptor layer 4 binds the analyte and interferents and the presence of these materials is detected by the transducer. The mechanism of the detection will vary depending on the nature of the transducer. However, the receptor layer 4 must be in communication with the transducer to allow the analyte and interferents to be detected by the transducer. For example, where the transducer 6 is an amperometric transducer or a conductimetric transducer, the receptor layer 4 must be in electronic communication with the transducer 6. The receptor layer 4 may be disposed directly on the transducer 6, or the receptor layer 4 may be proximal to the transducer 6 and electronic communication is established by the presence of an electrolyte or other electrically conductive material between the receptor layer 4 and the transducer 6. Where the transducer 6 is thermal transducer, the receptor layer 4 is in thermal communication with the transducer 6. This may again be by direct contact or the receptor layer 4 may be proximal to the transducer 6 and thermal communication is established by the presence of a thermally conductive material between the receptor layer 4 and the transducer 6.

The transducer 6 is itself preferably disposed on the substrate 5. The transducer 6 may be disposed on the surface of the substrate 5 or it may be disposed within the substrate 5. The transducer 6 and the receptor layer 4 may also constitute a single entity. For example, an electrode material may be screen-printed onto a suitable substrate 5. A polymer (forming the receptor material) and graphite (forming both the transducer 6 and a dispersed electrically conductive material within the receptor layer 4) may then be combined and screen-printed onto the electrode material. The sensor 1 may also comprise further transducers and receptor layers to detect further analytes. The substrate 5 is preferably a planar substrate. The substrate 5 may be composed of silicon (e.g. a silicon wafer), ceramic, glass, metal, plastics etc. Alternatively, the receptor layer 4 itself may sufficiently resilient to act as a substrate and a separate substrate 5 is not required.

FIG. 2A shows the receptor layer as a MIP. The unfilled triangles represent the binding sites for the analyte. The binding sites are provided by synthesising the MIP in the presence of the analyte to be detected, or a close structural analogue of the analyte, using well-known techniques, see WO 2005/075995 and WO 2006/120381.

As the MIP has been imprinted with the analyte or analogue, the MIP will interact with the target analyte more strongly than the interferents. For example, due to the imprinting process, the analyte may have an increased number of interaction points with the MIP in comparison with the interferents, which may only interact with the MIP via non-specific binding.

As shown in FIG. 2B, the sample 2 is brought in to contact with the receptor layer 4. When operating the transducer 6 at this time, both the analyte and the interferents will contribute to the recorded signal. The amplitude of this signal is typically equal to the sum of the signals produced by the presence of the target analyte (through both specific and non-specific binding) and the non-specific binding of the interferents.

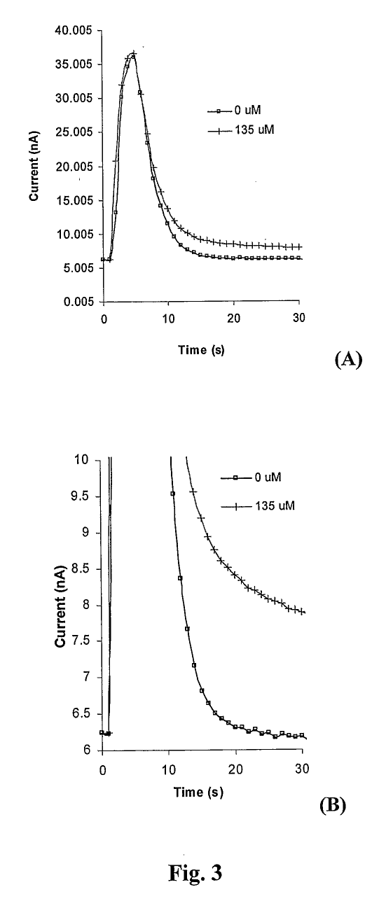

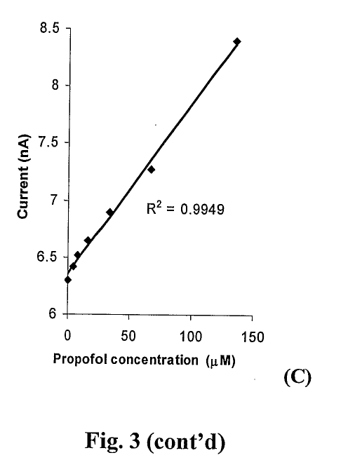

In a subsequent step, the receptor layer 4 (e.g. the MIP) is washed. The washing rapidly removes from the receptor both the weakly bound interferents and the target analyte which is weakly bound by non-specific binding, see FIG. 2C; the more strongly bound analyte which has bonded by specific bonding to the receptor is released from the receptor material more slowly. This washing step results in a rapid drop in the level of the signal recorded by the transducer, arising from the removal of the interferents from the receptor layer 4, followed by a longer tail, caused predominantly by the slow release of the analyte from the receptor layer 4. This tail continues as the bound analyte continues to be washed away from the receptor layer 4. A typical sensor trace is discussed hereinbelow with reference to FIG. 3.

The sensor of the present invention may be prepared by microfabricating a sensor chip and depositing a receptor layer on the transducer using the methodology discussed in WO 2005/075995 and WO 2006/120381. A sample potentially containing the analyte of interest in the presence of at least one interferent may then be introduced to the receptor layer and the analyte and interferents are allowed to bind to the receptor layer. The receptor layer having the bound analyte and interferents is then treated to remove selectively the at least one interferent. Measurements are taken from the transducer at varying times and the results are analysed with reference to a suitable calibration curve to determine the amount of analyte present in the sample.

The signal at a certain time following the start of the treatment step is recorded. This signal is then taken to be a measure of the concentration of the analyte in the sample. The concentration of analyte in the sample being analysed can be determined by measuring the signal recorded with the transducer a certain time after the treatment step has been started. The signal recorded at this time is indicative of the analyte concentration in the sample, as shown diagrammatically in FIG. 2C. After the initial removal of the weakly bound materials, the signal transduced is related to the concentration of the analyte present in the sample.

This approach therefore enables the discrimination of the analyte from the interferents and the detection and concentration measurement of the analyte in a complex sample containing one or more interferent(s). The time delay between the start of the washing step and the recoding of the signal depends, for example, on the type of sensor used, the material of the receptor layer, the thickness of the receptor layer and the geometry of the sensor. It can therefore be tailored to suit the particular application.

The treatment step will depend on the nature of the analyte, the interferents and the receptor layer itself. Suitable techniques include applying a change in potential, a change in pH or a change in temperature to the receptor layer, washing the receptor layer, irradiating the receptor layer, or a combination thereof. Preferably, the interferents are selectively removed by washing the receptor layer.

The fluid used in the washing step will of course also depend on the nature of the analyte, interferents and the receptor layer. For example, in a preferred embodiment, the receptor layer may be formed from a molecularly imprinted polymer, the analyte is the anaesthetic propofol and the interferents are uric acid and ascorbic acid. In this case, the aqueous washing liquid may conveniently be one of the aqueous flushing or calibrating fluids which are used in sensors of this type, e.g. phosphate buffer solution. See WO 99/62398 for a discussion of suitable calibrating fluids.

In another embodiment, the analyte may be removed by washing with an organic solvent, such as THF, acetonitrile or alcohol. Similarly, bound analytes and the interferents present may be distinguished by washing with an acidic or basic washing liquid.

The washing step may be performed by applying a separate washing liquid to the receptor layer, or simply by changing the chemical composition of the fluid already in contact with the receptor layer. Similarly, the change in pH may be achieved by washing with a washing liquid having different pH or by introducing and acid or base into the fluid already in contact with the receptor layer.

In one particular example of the invention a method is therefore provided for operating a sensor in a complex mixture comprising of the analyte to be measured, without the need for a protective layer over the receptor layer, by measuring the presence of the analyte bound in or on the layer after the weakly bound interferents have been removed.

Preferably, the receptor layer has a sufficient capacity for the analyte to allow multiple or continuous use of the sensor.

In another embodiment of the invention, the method further comprises, after the treatment step, the step of releasing the analyte from the receptor layer. Thus, the initial treatment step to release the interferents from the receptor layer 4 is followed by a regeneration step using an external stimulus of the type discussed hereinabove (i.e. by applying a change in potential, a change in pH or a change in temperature to the receptor layer, by washing the receptor layer, by irradiating the receptor layer, or a combination thereof, and preferably, by washing). This regeneration step will then release the analyte from the receptor material at a higher rate, resulting in a higher signal due to the analyte. This approach can be used to improve the signal to noise ratio and therefore the sensitivity of the measurement. It is particularly used to provide a (sudden) increase in the amount of analyte being released from the receptor layer in circumstances where either the release rate or the amount released in the first step is low. In the example of the propofol sensor discussed hereinabove, this subsequent removal of the propofol may be achieved by using a potential change. The propofol may also be removed by continuing to wash with the same washing fluid.

In a particularly preferred embodiment of the present invention, the sensor 1 is used for the measurement of propofol in a blood sample, which employs a MIP as the receptor layer. More preferably, the MIP is immobilised on top of an amperometric transducer.

Preferably, the sensor 1 is used to oxidise the propofol being released by the MIP. This can be achieved, for example, by operating the transducer as an amperometric transducer and applying a voltage of 0.35 V or larger between the working electrode and the reference electrode. By choosing this voltage carefully, i.e. just slightly above the level at which propofol can be oxidised, the oxidation of other species having a higher oxidation potential can be suppressed.

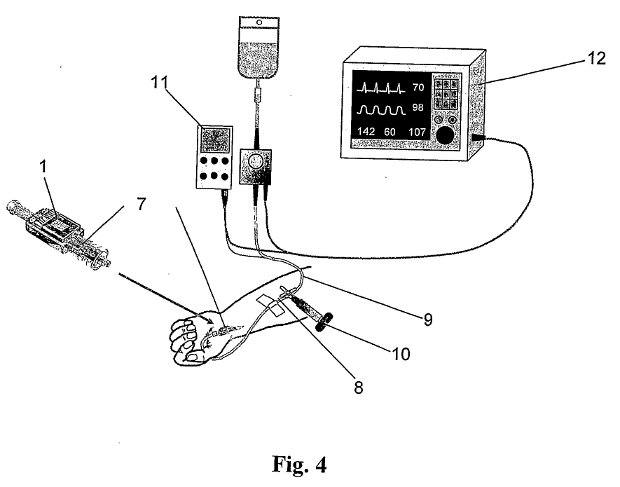

In use, the sensor 1 is typically incorporated into a sampling apparatus. The sampling apparatus comprises a housing coupled to a sampling port and incorporating the sensor as described herein, and a signal processing unit in electronic communication with the sensor. An example of such a system is shown in FIG. 4. The system is equipped with a housing 7 incorporating the sensor 1 coupled to a sampling port 8 in an intravascular line 9 above the sensor 1. A sampling device 10, for example a syringe, is coupled to the sampling port 8. Using the sampling device 10, the user will withdraw blood flushing it across the sensor 1 in order to take a measurement. After the measurement is completed, the blood may be flushed back into the patient or it may be flushed to waste. In another embodiment, the sensor can be incorporated into the intravascular-flushing line, for example, along with one or more other sensors, such as a pressure sensor. Samples may be taken either periodically, regularly, event-driven, on demand or following a user intervention.

The sensor 1 is connected to a local display and signal processing unit 11 which may be connected to a patient monitoring device 12. The sensor 1 is also connected to the housing 7 electronically using techniques known in the art.

In addition to the system described above, the sensor may be employed in a range of other sensing systems, known to those skilled in the art. For example, rather than being directly connected to the patient, a sample may be taken from the patient and transported to and injected into an analyser, into which the sensor is integrated, for sample analysis.

In addition to providing detection and measurements of markers, substances or drugs, the sensor of the present invention provides feedback for the treatment of the patient based on the results of the analysis made. This feedback may be provided either directly to the user or it may be part of a closed-loop control system including the device administering the treatment to the patient. One particular example is a sensor for an anaesthetic agent, such as propofol, which measures the concentration of the anaesthetic agent in one or more bodily fluids or body compartments, e.g. blood or blood plasma, and based on these measurements directs, either directly or the user, the subsequent delivery of the anaesthetic agent, e.g. by controlling the rate of delivery to the patient via a syringe pump.

The sensor may also be used with systems which monitor other parameters which characterise the health of a patient, monitor particular markers indicating disease states or direct the patient's treatment, e.g. blood gases, pH, temperature etc.

### Example

The present invention will now be described with reference to the following examples which are not intended to be limiting.

### Example 1

### Sensor Preparation

A sensor was prepared by microfabricating a sensor chip and immobilising a MIP on the transducer using the methodology discussed in WO 2005/075995 and WO 2006/120381.

Specifically, to ensure the robust attachment of the MIP layer to the electrode surface as well as gain control over the polymer formation, the polymerisation initiator was firstly anchored to the electrodes. Clean oxidised platinum electrodes were exposed to 3% 3-aminopropyl triethoxysilane in dry toluene for 3 hours in order to introduce amino functionalities at the sensor surface. The polymerisation initiator 4,4′-azobis(cyanovaleric acid) was then covalently attached to the amino layer via carbodiimide coupling by exposing the derivatised sensor to a mixture of 20 mM 4,4′-azobis(cyanovaleric acid), 17 mM N-(3-dimethylaminopropyl)N′-ethylcarbodiimide and 28 mM 1-hydroxybenzotriazole. The reaction was left to take place at room temperature in the dark for 5 hours. The derivatised electrodes were rinsed thoroughly with acetone to remove any non-covalently bound initiator, and finally dried in a stream of nitrogen. The derivatised sensors were kept in the dark and used on the same day.

The derivatised sensors were immersed in 200 μL of an oxygen-free MIP pre-polymerisation mixture consisting of 50 mg of propofol, 210 mg of DEAEM (monomer), 1.3 g of ethylene glycol dimethacrylate (cross linker) dissolved in 1.55 g of dimethylformamide. The vessel was flushed with nitrogen and finally sealed with a quartz glass slide. A UV light guide connected to a UV source was then placed on top of the quartz slide and actuated for 5 minutes. The sensor was finally taken out of the vessel and rinsed with methanol, washed with 5 mL of 0.1 M HCl/20% methanol, rinsed with water, and washed with 5 mL of 0.1 M NaOH/20% methanol, rinsed with water, and finally blow dried in a stream of compressed air. Imprinted polymer films typically 45 nm thick, as characterised by atomic force microscopy, were obtained.

### Example 2

### Sensor Evaluation

Samples of phosphate-buffered saline containing the anaesthetic propofol in the presence of the interferents uric acid and ascorbic acid were introduced to the MIP and the analyte and interferents were allowed to bind to the MIP. The MIP having the bound analyte and interferents was then washed with phosphate-buffered saline (140 mM NaCl, 10 mM phosphate). Measurements were taken using an amperometric transducer at varying times and the results are shown in FIG. 3.

FIG. 3A shows the signal of the interferents alone with no propofol (labelled “0 uM”), and the interferents in the presence of 135 μM of propofol (labelled “135 uM”). FIG. 3B shows a region of graph (A) in greater detail illustrating the sharp drop in signal due to the rapid removal of the weakly bound interferents and the slow signal decrease in the presence of propofol. In this example, the signal 30 s following the start of the washing step was taken to be a measure of the concentration of the analyte, propofol, in the sample. FIG. 3C shows a linear calibration curve for propofol concentrations of 4.2, 8.4, 16.9, 33.5, 67.5 and 135 μM in the presence of constant levels of uric and ascorbic acid. The concentration of propofol in the unknown sample can thus be determined with reference to the calibration curve.

1. A method for detecting an analyte in the presence of at least one interferent in a sample comprising the steps of
providing a sensor having a transducer and a receptor layer in communication with the transducer, wherein the receptor layer comprises a material for absorbing the analyte;
exposing the receptor layer to the sample;
treating the receptor layer to remove selectively the at least one interferent; and
measuring the signal from the transducer.

2. A method as claimed in claim 1, wherein the sensor further comprises a substrate.

3. A method as claimed in claim 2, wherein the transducer is disposed on the substrate.

4. A method as claimed in any preceding claim 2, wherein the sensor further comprises a confinement structure, the confinement structure comprising a first limiting structure defining a first interior space, and wherein the transducer and the receptor layer are disposed in the first interior space.

5. A method as claimed in claim 4, wherein the first limiting structure is a continuous structure.

6. A method as claimed in claim 4, wherein the first limiting structure is annular.

7. A method as claimed in claim 1, wherein the sensor further comprises a channel to contain the sample.

8. A method as claimed in claim 1, wherein the sample is a fluid sample.

9. A method as claimed in claim 8, wherein the fluid sample is a bodily fluid.

10. A method as claimed in claim 1, wherein the receptor layer comprises a synthetic polymer.

11. A method as claimed in claim 10, wherein the receptor layer comprises a molecularly imprinted polymer.

12. A method as claimed in claim 1, wherein the transducer is an amperometric transducer, a potentiometric transducer, a conductimetric transducer, an optical transducer, a gravimetric transducer, a surface-acoustic wave transducer, a resonant transducer, a capacitive transducer or a thermal transducer.

13. A method as claimed in claim 1, wherein the analyte is propofol, the sample is a bodily fluid and the washing step is performed with an aqueous liquid.

14. A method as claimed in claim 1, wherein the receptor layer has a sufficient capacity for the analyte to allow multiple or continuous use of the sensor.

15. A method as claimed in claim 1, wherein treating the receptor layer to remove selectively the at least one interferent is performed by applying a change in potential, a change in pH or a change in temperature to the receptor layer, by washing the receptor layer, by irradiating the receptor layer, or a combination thereof.

16. A method as claimed in claim 15, wherein treating the receptor layer is by washing the receptor layer.

17. A method as claimed in claim 1, wherein the method further comprises, after treating the receptor layer to remove selectively the at least one interferent, the step of releasing the analyte from the receptor layer.

18. A method as claimed in claim 17, wherein the analyte is released by applying a change in potential, a change in pH or a change in temperature to the receptor layer, by washing the receptor layer, by irradiating the receptor layer, or a combination thereof.

19. A method as claimed in claim 18, wherein the analyte is released by washing the receptor layer.

20. A method as claimed in claim 18, wherein the analyte is propofol, the sample is a bodily fluid and the analyte is released by applying a change in potential.

21. A method according to claim 1 wherein the receptor layer comprises a receptor material and a dispersed electrically conductive material.
